(19) United States
(12) Patent Application Publication
(10) Pub. No.: US 2008/0279804 A1
(43) Pub. Date: Nov. 13, 2008

(54) Hair styling formulation and method of use

(76) Inventors: Tiffany L. Parker, Norwalk, CT (US); Jeffrey J. Miles, Southbury, CT (US)

Correspondence Address: Melvin I. Stoltz, 51 Cherry Street, Milford, CT, 06460, US

(21) Appl. No.: 12/148,984

(22) Filed: Apr. 24, 2008

Related U.S. Application Data
(60) Provisional application No. 60/926,571, filed on Apr. 27, 2007.

(51) Int. Cl. A61K 8/25; A61K 8/72

(52) U.S. Cl. 424/70.11; 424/70.1; 510/119

(57) ABSTRACT

In accordance with the present invention, a highly effective, hair styling foundation composition and a method of use are achieved. By employing the present invention, the hair styling foundation composition enhances the performance and efficacy of additional compositions, such as styling polymers and conditioning treatments, while also creating a protective film on the hair fibers. In this way, a highly effective and beneficial hair treatment is realized.

### RELATED APPLICATIONS

This application is related to U.S. Provisional Patent Application Ser. No. 60/926,571, filed Apr. 27, 2007 entitled HAIR STYLING FORMULATION AND METHOD OF USE.

### TECHNICAL FIELD

This invention relates to a hair care products and, more particularly, to hair styling foundations for use with hair care products.

### BACKGROUND ART

The composition of hair has been well studied and understood. Hair strands are essentially composed of three layers: a center medulla surrounded by the cortex and an exterior cuticle. The main component of hair is a protein called Keratin, which forms the primary structure and physical properties of the hair. The remaining materials consist primarily of lipids, melanin and amino acids.

The cuticle is the exterior barrier between the hair and the environment. It consists of overlapping scale-like cells which are subject to damage from shampooing, chemical treatments, combing and environmental exposure such as UV irradiation. In the intercellular spaces between cuticle cells and between the cuticle and the cortical cells is a substance known as the Cell Membrane Complex (CMC). The CMC is composed of lipids, including 18-Methyl Eicosanoic Acid (18-MEA), and provides cell fluidity and hydrophobicity.

As hair fibers become damaged, the ionic charge of the hair's surface becomes more negatively charged. Cuticle cells start to lift off the surface of the surface, cracking and abrading with more damage. Hair becomes less hydrophobic, retaining less moisture and becoming less supple.

Many hair products have been developed to counteract the effects of damage on the hair. Often these products deposit a positively charged quaternized conditioning material or a non-ionic silicone to replenish the hydrophobic barrier and neutralize the anionic charge. However, these systems are limited because the deposited materials are removed with successive shampooing.

Another category of products focuses not on counteracting the effects of damage but on depositing polymers for the purpose of improving the style and style retention of the hair. These products are also limited in their efficaciousness. Not only does shampooing remove these products but high humidity and time impact the style retention when using a traditional styling product

Therefore, it is a principal object of the present invention to provide a hair care product which is able to be easily applied to the hair and provides a long lasting foundation which binds to the surface of the hair fibers and enables additives applied to the hair to be inherently bound therewith.

Another object of the present invention is to provide a hair care product having the characteristic features described above which is capable of increasing the concentration in which materials are bound to the hair while also increasing the duration of time in which these materials remain on the hair surface.

Another object of the present invention is to provide a hair care product having the characteristic features described above which functions to prevent damage to the hair fibers.

Other and more specific objects will in part be obvious and will in part appear hereinafter.

### THE DRAWINGS

For a fuller understanding of the nature and objects of the invention, reference should be had to the following detailed description taken in connection with the accompanying drawings, in which:

FIG. 1 is a mass spectrograph of C10-40 Isoalkylamidopropylethyldimonium Ethosulfate;

FIG. 2 is an ion chromatogram for molecular ion m/z 397; and

FIG. 3 is a chart showing the ratio of reflectance values achieved using the present invention.

### DETAILED DISCLOSURE

By employing the present invention, the prior art difficulties and inabilities are overcome, and a highly effective, hair styling foundation composition and a method of use are realized. By employing the present invention, a protective film is applied to the hair fibers which prevents damage to the hair fibers while also enabling additional treatments applied to the hair, such as styling polymers and conditioning treatments, to be employed with greater efficacy and enhanced results. In this way, a highly effective and beneficial hair treatment is realized.

In accordance with the present invention, the desirable and previously unattainable results are realized by applying a foundation composition to the surface of the hair fibers in a sufficient quantity to establish a protective film on the hair surfaces. It has been found that by employing the foundation composition of the present invention, the quantity of the anionic sites on the hair surface are increased, and a protective film over the surface is created, reducing the impact of degradation. In addition, it has also been found that by employing the foundation composition of the present invention additional benefits are realized which enable styling compositions, conditioning agents, and the like, that are subsequently applied to the hair to experience increased efficacy and surface deposition.

The foundation composition binds to the hair surface due to charge and ion interaction. It is believed that the application of the foundation composition of the present invention to the hair fibers causes the quantity of anionic sites on the hair surface to be substantially increased. Once the foundation composition has been applied to the hair surface, any supplemental product or products desired for application to the hair fibers may be employed with greater efficacy. Typically, the supplemental products are in the form of a hair enhancing formulations, such as styling and conditioning polymers, formulations, and/or treatments. Preferably, the supplemental products are cationic compositions which interact with the anionic sites created by foundation composition of the present invention to result in substantially increased absorption and adherence of the supplemental products on the hair surface.

It has been found that the benefits provided by the foundation composition of the present invention are achieved regardless of whether the foundation composition is left on the hair surface or rinsed off prior to the application of the supplemental products. In this regard tests conducted clearly demonstrate that the foundation composition is capable of increasing the absorption and retention of the supplemental products when the foundation composition has been applied to the hair surface and left thereon, as well as when the foundation composition has been applied to the hair surface and rinsed off prior to the application of the supplemental products.

Furthermore, it has also been found that the beneficial effects provided by the foundation composition of the present invention are realized even when the supplemental products are applied to the hair surface prior to the application of the foundation composition. In this regard, it has been found that the supplemental product applied to the hair fibers prior to the foundation composition preferably comprises a cationic substance, a substance capable of binding to the hair fibers due to its own chemistry, and/or a product depositing proteins. After the application of the supplemental product, the foundation composition of the present invention is applied to the hair surface and either left on or if desired, rinsed off.

In both operations, a protective layer is achieved on the hair surface, along with binding of the deposited material on the hair surface. In addition, it has also been found that the foundation composition of the present invention can be applied to the hair surface by itself which results in a protective layer being achieved on the hair surface along with the binding of the hair's internal protein and composition.

In order to attain the beneficial results detailed above, it has been found that the foundation composition of the present invention preferably comprises an aqueous solution of sodium magnesium silicate. In this regard, between about 0.05% and 5% by weight based upon the weight of the entire composition comprises sodium magnesium silicate with water forming the balance. In addition, in the preferred formulation, a preservative is preferably employed which comprises between about 0.2% and 1.5% by weight based upon the weight of the entire composition.

If desired, other additives can be incorporated into the foundation composition in order to provide further enhancements thereto. These additives include thickeners, suspension aids, silicones, humectants, solubilizers, fragrances, calcium ion source, and pH adjusters. If desired, other additives can be employed without departing from the scope of this invention. In Table I, an overall formulation incorporating the preferred additives in addition to the principal components is fully detailed, along with the preferred quantity range employed for each ingredient.

TABLE I
Hair Styling Foundation

| Name | % w/w | Function |
|---|---|---|
| Water/Aqua/Eau | q.s. | Solvent |
| Sodium Magnesium Silicate | 0.05-5.00 | Binder, film former, anionic |
| | | site enhancer |
| Preservative | 0.05-1.50 | Preservative |
| Thickener/Suspension Aid | 0.00-5.00 | Stabilizer |
| Silicones | 0.00-10.00 | Shine enhancer, emollient |
| Humectants | 0.00-15.00 | Shine enhancer, emollient |
| Solubilizer | 0.00-2.50 | Solubilizer |
| Fragrance | 0.00-1.00 | Fragrance |
| pH Adjuster | 0.00-3.00 | pH adjustment |
| Calcium Ion Source | 0.00-2.00 | Binder |
| Emulsifier | 0.00-8.00 | Emulsifier |

It has been found that the hair styling foundation formulation of the present invention can be applied directly to the hair, as detailed herein, or if desired, can be formulated as a spray composition or a shampoo composition for application to the hair. In Table II, the preferred composition for the hair styling foundation formulated as a spray composition is provided, while Table III details the preferred composition for the hair styling foundation formulated as a shampoo composition

TABLE II
Hair Styling Foundation As Spray Composition

| Name | % w/w | Function |
|---|---|---|
| Water/Aqua/Eau | q.s. | Solvent |
| Sodium Magnesium Silicate | 0.05-5.00 | Binder, firm former, anionic |
| | | site enhancer |
| Preservative | 0.05-1.5 | Preservative |
| Thickener/Suspension Aid | 0.00-5.00 | Stabilizer |
| Silicones | 0.00-10.00 | Shine enhancer, emollient |
| Humectants | 0.00-15.00 | Shine enhancer, emollient |
| Solublizer | 0.00-2.50 | Solublizer |
| Fragrance | 0.00-1.00 | Fragrance |
| Protein, Vitamins, | 0.00-5.00 | Functional ingredients |
| Herbals, UV absorbers | | |
| Polymers | 0.00-10.00 | Film former |
| Calcium Ion Source | 0.00-2.00 | Binder |
| pH Adjuster | 0.00-3.00 | pH Adjustment |

TABLE III
Hair Styling Foundation As Shampoo Composition

| Name | % w/w | Function |
|---|---|---|
| Water/Aqua/Eau | q.s. | Solvent |
| Sodium Magnesium Silicate | 0.05-5.00 | Binder, firm former, |
| | | anionic site enhancer |
| Primary Surfactant | 5.00-20.00 | Surfactant |
| Secondary, Tertiary and | 0.20-20.00 | Surfactant |
| any additional Surfactants | | |
| Thickener/Suspension Aid | 0.00-10.00 | Stabilizer, Thickener |
| Silicones | 0.00-10.00 | Shine enhancer, emollient |
| Humectants | 0.00-15.00 | Shine enhancer, emollient |
| Preservative | 0.05-1.5 | Preservative |
| Fragrance | 0.00-1.00 | Fragrance |
| Protein, Vitamins, Herbals, UV | 0.00-5.00 | Functional ingredients |
| absorbers | | |
| Polymers | 0.00-10.00 | Film Former |
| Calcium Ion Source | 0.00-2.00 | Binder |
| pH Adjuster | 0.00-3.00 | pH Adjuster |

In the foregoing formulations, any desired additive coming within the scope of the additives detailed in the formulations can be employed. However, in this regard, it has been found that the preferred preservatives comprise one or more selected from the group consisting of DMDM Hydantoin, Iodopropynyl Butylcarbamate, Methylparaben, Propylparaben, Methylchloroisothiazolinone, Methylchloroisothiazolinone, Diazolidinyl Urea, and Imidazolidinyl Urea. In addition, DMDM Hydantoin is preferred.

Furthermore, the thickeners/suspension aids preferably comprise one or more selected from the group consisting of Carbomer, Acrylamide/Sodium Acryloyldimethyltaurate/Acrylic Acid Copolymer, Acrylates/Ceteth-20 Itaconate Copolymer, Sodium Polyacrylate, Polyacrylamide, Hydrogenated Polydecene, Hydroxyethylcellulose, Xanthan Gum, Carrageenan, and Guar, with carbomer or Acrylamide/Sodium Acryloyldimethyltaurate/Acrylic Acid Copolymer being preferred. In addition, the silicones are preferably comprise one or more selected from the group consisting of PEG/PPG-15/15 Dimethicone, PEG/PPG-18/18 Dimethicone, PEG-12 Dimethicone, PEG/PPG-17/18 Dimethicone, Lauryl PEG/PPG-18/18 Methicone, PEG/PPG-20/6 Dimethicone, PEG/PPG-4/12 Dimethicone, PEG/PPG-20/20 Dimethicone, Dimethicone, Amodimethicone, and Polysilicone-15, with PEG/PPG-15/15 Dimethicone being preferred. The humectants preferably comprise one or more selected from the group consisting of Glycerin, Sorbitol, Propylene Glycol, Butylene Glycol, Rice Oil Glycereth-8 Esters, Acetamide MEA, Hydroxyethyl Urea, and Methyl Gluceth-20, with glycerin being preferred. The emulsifiers preferably comprise one or more selected from the group consisting of Cetyl Alcohol, Stearic Acid, Glyceryl Stearate, PEG-100 Stearate, Stearyl Alcohol, Cetearyl Alcohol, Cetyl Esters, Palmitic Acid and Lecithin, with Cetyl Alcohol being preferred. The solubilizers preferably comprise one or more selected from the group consisting of PPG-5-Ceteth-20, Trideceth-6, Laureth-7, Polysorbate 20, Polysorbate 40, Polysorbate 80, PEG-40 Hydrogenated Castor Oil, and PEG-60 Hydrogenated Castor Oil, with PPG-5-Ceteth-20 being preferred. The pH adjustors preferably comprise one or more selected from the group consisting of Aminomethyl Propanol, Triethanolamine, Citric Acid, EDTA, Disodium EDTA, Tetrasodium EDTA, Trisodium EDTA, Sodium Hydroxide, and Phosphoric Acid, with Aminomethyl Propanol or citric acid being preferred.

In the formulation of the hair styling foundation as a spray composition and/or shampoo, additional additives are incorporated into the compositions. In this regard, the protein, vitamins, herbals, and UV absorbers preferably comprise one or more selected from the group consisting of Hydrolyzed Keratin, Hydrolyzed Soy Protein, Hydrolyzed Wheat Protein, Keratin, Bletilla Striata Root Extract, Euterpe Oleracea Fruit Oil, Squalane, Tocopheryl Acetate, Retinyl Palmitate, Tocopherol, Arganine, Cystine, Benzophenone-2, Ethylhexyl Methoxycinnamate, Allantoin, with hydrolyzed keratin being preferred. The polymers preferably comprise one or more selected from the group consisting of PVP, Vinyl Caprolactam/VP/Dimethylaminoethyl Methacrylate Copolymer, Octylacrylamide/Acrylates/Butylaminoethyl Methacrylate Copolymer, Sodium Polystyrene Sulfonate, Acrylates/Octylacrylamide Copolymer, VP/VA Copolymer, Polyquaternium-11, Polyquaternium-46, VP/DMAPA Acrylates Copolymer, Polyurethane-18, Polyacrylate-22, and VP/Methylacrylamide/Vinyl Imidazole Copolymer, with PVP or Octylacrylamide/Acrylates/Butylaminoethyl Methacrylate Copolymer being preferred. Finally, the calcium ion source preferably comprises one or more selected from the group consisting of Calcium Chloride, Calcium Citrate, Calcium Sulfate, and Calcium, with calcium chloride being preferred.

In the formulation of the hair styling foundation as a shampoo, the composition also incorporates a primary surfactant which preferably comprises one or more selected from the group consisting of Sodium Laureth Sulfate, Ammonium Laureth Sulfate, Sodium Lauryl Sulfate, Ammonium Lauryl Sulfate, Cocamidopropyl Betaine, TEA-Lauryl Sulfate, Ammonium Xylenesulfonate, Disodium Laureth Sulfosuccinate, Disodium Cocoamphodiacetate, Cocamidopropyl Hydroxysultaine, Sodium Lauroyl Sarcosinate, and Disodium Cocoyl Glutamate, with Sodium Laureth Sulfate being preferred. In addition, a secondary, tertiary, and additional surfactants may be employed which preferably comprise one or more selected from the group consisting of Cocamidopropyl Betaine, Sodium Laureth Sulfate, Ammonium Laureth Sulfate, Sodium Lauryl Sulfate, Ammonium Lauryl Sulfate, Cocamide MEA, Cocamide DEA, TEA-Lauryl Sulfate, Ammonium Xylenesulfonate, Disodium Laureth Sulfosuccinate, and Disodium Cocoamphodiacetate, Cocamidopropyl Hydroxy-sultaine, Sodium Lauroyl Sarcosinate, Disodium Cocoyl Glutamate, with Cocamidopropyl Betaine being preferred.

In the preferred application method for employing the foundation composition of the present invention, the foundation composition is layered onto the hair fibers in order to thoroughly cover the hair fibers substantially in their entirety. Similarly, the supplemental products applied to the hair fibers are also layered onto the hair fibers in a substantially similar manner. In this way, it has been found that the efficacy of the present invention is fully realized.

As detailed above, sodium magnesium silicate comprises the principal ingredient of the foundation composition of the present invention. In this regard, any desired source for sodium magnesium silicate can be employed, which is a colloidal synthetic layered silicate. Sodium magnesium silicate is commercially available under the trade name LAPONITE, which is distributed by Rockwell Specialties Group of Princeton N.J.

The invention accordingly comprises the several steps and the relation of one or more such steps with respect to each of the others and the composition possessing the features, properties, and relation of components, all as exemplified herein, with the scope of the invention being indicated in the claims.

### BEST MODE FOR CARRYING OUT THE INVENTION

In order to substantiate the efficacy of the hair styling foundation of the present invention, numerous tests were conducted in which hair tresses were treated with the hair styling foundation of the present invention using a wide variety of alternate procedures, and compared to hair tresses treated in the same way with a control solution. The following examples are presented in order to fully demonstrate a highly effective results achieved by the hair styling foundation of the present invention.

By reviewing the following examples, the ability of the hair styling foundation of the present invention to produce the desired results is clearly established. However, it is to be understood that the following examples are intended as a teaching of the best mode for carrying out the present invention and are not intended to limit the breadth of this discovery.

In applying the hair styling foundation of the present invention to the hair surface, the hair styling composition containing styling polymers and cationic conditioning treatments was subsequently layered onto the surface of the hair fibers. In another method, all additional compositions, including styling polymers and cationic conditioning treatments were also layered onto the hair surface either separately or at the same time as the active material. In this way, only a single step is required. Depending on the active material, it will be deposited under the Sodium Magnesium Silicate or will be bound to the Sodium Magnesium Silicate or a combination of both modes of action. For example, UV absorbers can be used in the same step as Sodium Magnesium Silicate with no detrimental impact on the UV absorbers' performance. By employing either the layering process or the simultaneous process, increased deposition of all active ingredients on the hair fibers is attained.

As detailed below, in conducting a wide variety of tests, numerous hair tresses were treated with the hair styling foundation formulation of the present invention as well as with a control solution. In addition, the hair tresses were treated with various cationic styling polymers. Curl droop and retention were evaluated over time for each test sample. As detailed below, the hair tresses treated with the hair styling foundation formulation of the present invention retained their curl longer than the control.

Furthermore, alternate methods for applying the hairstyling foundation formulation of the present invention were also tested. These various methods included (1) applying the hairstyling formulation and immediately rinsing it off, (2) applying the hairstyling formulation and immediately applying the hairstyling polymer and/or cationic conditioning agents followed by rinsing, (3) applying the hairstyling formulation and leaving it on the hair fibers for three minutes, followed by the application of the hair styling polymers and/or cationic conditioning agents followed by rinsing, (4) applying the hairstyling formulation and immediately rinsing it off, followed by the application of the hairstyling polymers and/or cationic conditioning agents, (5) intermixing the hair styling polymers and/or cationic conditioning agents with the hair styling formulation and applying the combined composition in a single application step, (6) applying the cationic conditioning agents prior to the hairstyling formulation, rinsing it off, applying hairstyling formulation and rinsing it off, and (7) applying the cationic conditioning agent prior to the hairstyling formulation, rinsing it off, applying the hairstyling formulation and leaving it on. As detailed below, the application and use of the hairstyling foundation formulation of the present invention significantly increased the deposition, retention, and efficacy of the additional styling polymers and conditioning agents regardless of the application methods employed.

Mass spectral analysis was done on hair extracts to confirm the presence and amount of a specific cationic conditioning active. The cationic active in these experiments was a quaternary ammonium derivative of integral hair lipid 18-MEA called C10-40 Isoalkylamidopropylethyldimonium Ethosulfate, see below. These experiments consisted of treating hair tresses with various product combinations, as detailed below, followed by multiple extractions in varying organic solvents, and then analysis by mass spectrometry.

Molecular structure for C10-40 Isoalkylamidopropylethyldimonium Ethosulfate

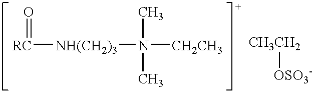

Cationic derivative of naturally occurring integral lipid 18-MEA on hair consists primarily of a mixture of C16-C22 derivatives with lesser amounts of lower and higher carbon chain derivatives present.

Hair tresses were either treated with Sodium Magnesium Silicate in varying forms including shampoo, rinse-off treatments and leave-on treatments, or a control. A cationic material was then applied to the hair tresses, also in varying forms including leave-on and rinse-off treatments. Controls were prepared that included untreated tresses, tresses treated only with the cationic conditioning treatment without the foundation composition, and tresses treated with the foundation composition only.

The extraction procedure was as follows: Approximately ½″ of hair was cut from the ends of each hair tress and accurately weighed to equal approximately 0.3 g. The cuttings from each tress were transferred into separate 125 ml Erlenmeyer flasks and 30 ml of extraction solution#1 was added to each flask. All the flasks were covered and swirled several times and allowed to extract for at least two hours, all extractions for an equal time. The extracts were then poured off into separate pear shaped flasks and set aside. 30 ml of extraction solution #2 was then added to each Erlenmeyer flask containing the different hair cuttings, covered, swirled several times, and allowed to extract again for a minimum of two hours. These extracts were then poured off into the same pear shaped flasks that collected the first extract from each Erlenmeyer flask. This process was continued for three more extractions so that the total extract volume for each hair tress equaled 150 ml. The extracts were then concentrated to dryness in a rotary evaporator and redissolved in 10 ml of methanol. The concentrated extracts were then filtered through a 0.45 micron syringe filter and prepared for analysis by mass spectrometry.

The second part of this experiment involved the identification and quantification of the cationic conditioning active, C10-40 Isoalkylamidopropylethyldimonium Ethosulfate, which was extracted from these hair tresses. The analytical technique used for this was atmospheric pressure electrospray ionization mass spectrometry with a single quadropole analyzer and using flow injection. The instrument used was an Agilent 1946D Single Quadrupole MSD set up with an electropspray ionization source coupled to an Agilent 1100 Liquid Chromatograph.

Method conditions were optimized by preparing solutions of the raw material, C10-40 Isoalkyamidopropylethyldimonium Ethosulfate, and flow injecting them into the mass spectrometer to see the ionization pattern. Because this material is a mixture of different carbon chain derivatives, a distribution of molecular ions was produced, and one particular molecular ion was chosen to monitor for quantification, see below. The molecular ion chosen was m/z 397 representing the C18 derivative and the following conditions were set to perform the analysis.

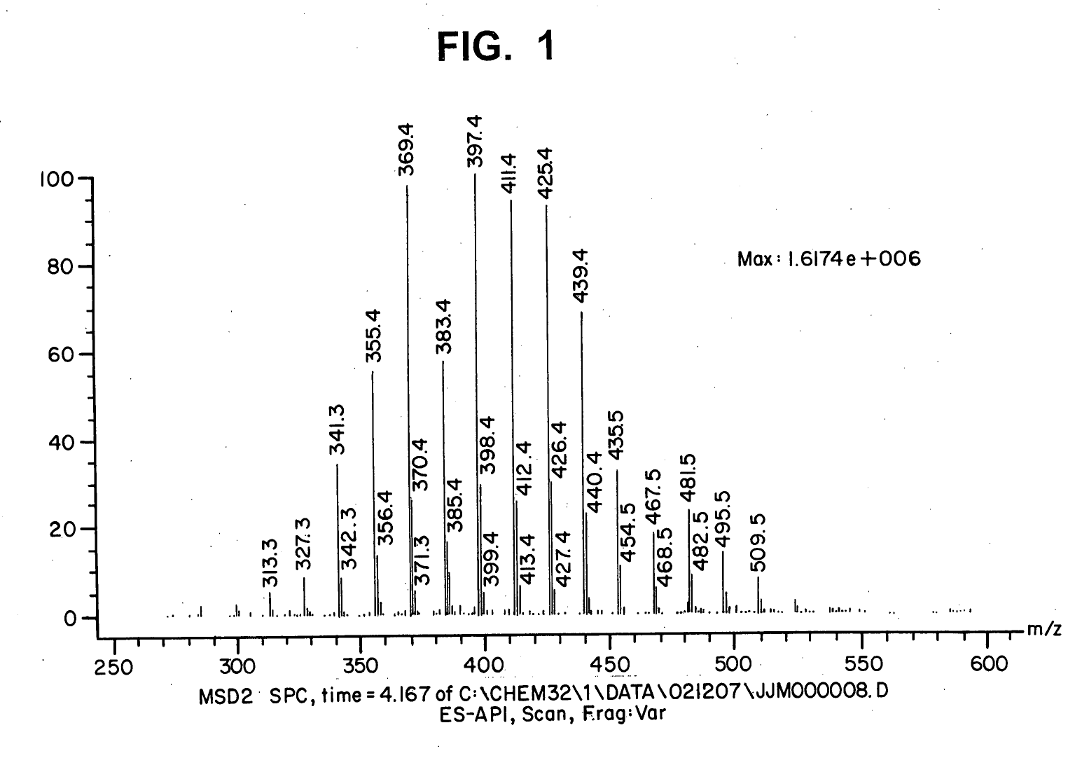

In FIG. 1, the mass spectrograph is provided for a C10-40 Isoalkylamidopropylethyldimonium Ethosulfate standard showing strong molecular ions at m/z 369, 383, 397, 411, 425, and 439 representing C16, C17, C18, C19, C20, and C21 derivatives respectively.

The following instrumental conditions were set to perform the analysis

LC Conditions:
Flow Rate=0.5 ml/min
Injection Volume=20 μl
Mobile Phase=90% Methanol
   10% 50 mM Ammonium Acetate Buffer @ pH 4.0

MSD Conditions:
Select Ion Monitoring (SIM)=m/z 397
Drying Gas Flow=11 l/min
Gas Temperature=350° C.
Nebulizer Pressure=40 psig
Fragmentor Voltage=120
Capillary Voltage=4000

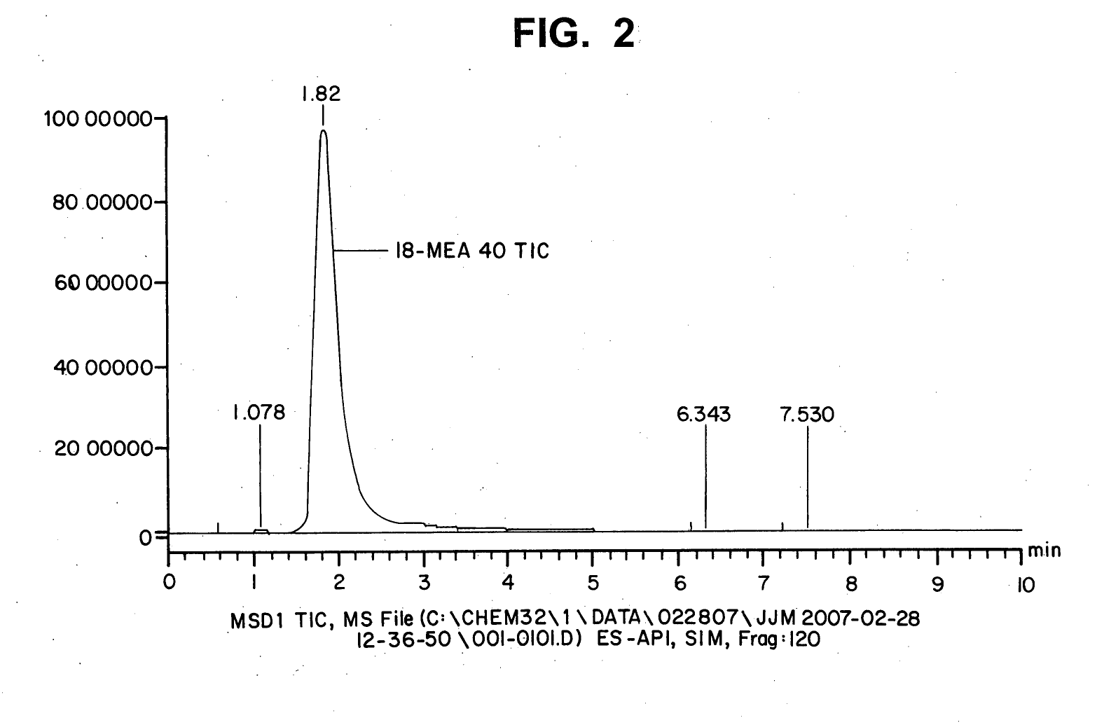

The resulting ion chromatogram for molecular ion m/z 397 was used to quantify the amount of cationic deposition on each tress versus a control. The response factor for each hair extract is ratioed against the response factor for an untreated hair extract to determine the relative amount of C10-40 Isoalkylamidopropyethyldimonium Ethosulfate deposited. In FIG. 2, the results of the following equation are provided.

$$\begin{matrix}\% C 10 - 40 Isoalkylamidopropyl \text{-} \\ ethyldimonium Ethosulfate\end{matrix} = \frac{( Sample Peak Area / Sample Conc , mg \text{/} ml )}{( Untreated Peak Area / Untreated Conc , mg \text{/} ml )}$$

To evaluate the alternate methods of application, hair tresses were tested utilizing multiple hair types and conditions of treatment application.

### Experiment 1:

In this test, medium brown hair tresses were washed with a 5% active solution of Sodium Laureth Sulfate (SLES) in water to remove any substances from the hair surface. An untreated control was designated, dried and set aside. A second control tress was treated with one gram of a 1% solution of C10-40 Isoalkylamidopropylethyldimonium Ethosulfate, rinsed with water at 38° C. (+/2.5° C.) at a flow rate of 60 ml/minute for fifteen seconds and dried.

The remaining tresses then underwent two types of treatment applications. In the first method of application, a hair tress was treated with one gram of the foundation composition of the present invention which was left on for three minutes. The tress was subsequently treated with one gram of a 1% solution of C10-40 Isoalkylamidopropylethyldimonium Ethosulfate, immediately rinsed with water as detailed above for fifteen seconds and dried.

In the second method of application, a hair tress was treated with one gram of the foundation composition of the present invention and immediately rinsed with water as detailed above for fifteen seconds. The tress was then treated with one gram of a 1% solution of C10-40 Isoalkylamidopropylethyldimonium Ethosulfate, immediately rinsed with water as detailed above for fifteen seconds and dried.

All hair tresses were submitted for mass spectral quantification of C10-40 Isoalkylamidopropylethyldimonium Ethosulfate as detailed above. The results are in Table IV and Table V.

TABLE IV
Injection Series #1 (4 injections of each extract Day 1)

| | | | | | % C10-40 |
|---|---|---|---|---|---|
| | | Injection | | | Isoalkyl- |
| | | Concentration, | | | amidopropyl- |
| | g of Hair | mg Hair | | Average | ethyldimonium |
| Description | Extracted | Extracted/ml | Peak Area | Peak Area | Deposited |
| Untreated Control | 0.2679 | 26.7900 | 26697.1000 | 14939.80 | *1.00 |
| | | | 12949.8000 | | |
| | | | 16156.4000 | | |
| | | | 15713.2000 | | |
| Control: C10-40 | 0.2222 | 22.2200 | 28178.1000 | 29240.95 | 2.36 |
| Isoalkylamidopropyl- | | | 30874.9000 | | |
| ethyldimonium | | | 31003.8000 | | |
| Ethosulfate Rinsed | | | 26907.0000 | | |
| Treatment Method I | 0.1688 | 16.8800 | 32516.2000 | 33568.98 | 3.57 |
| | | | 31889.9000 | | |
| | | | 38544.8000 | | |
| | | | 31325.0000 | | |
| Treatment Method II | 0.3102 | 31.0200 | 47633.7000 | 45359.70 | 2.62 |
| | | | 42623.6000 | | |
| | | | 43393.6000 | | |
| | | | 47787.9000 | | |

*Normalized to 1.00. All other results are relative to this control

TABLE V
Injection Series #2 (3 injections of each extract) Day 2

| | | | | | % C10-40 |
|---|---|---|---|---|---|
| | | Injection | | | Isoalkyl- |
| | | Concentration, | | | amidopropyl- |
| | g of Hair | mg Hair | | Average | ethyldimonium |
| Description | Extracted | Extracted/ml | Peak Area | Peak Area | Deposited |
| Untreated Control | 0.2679 | 26.7900 | 47458.144 | 51629.837 | *1.00 |
| | | | 52106.385 | | |
| | | | 55324.981 | | |
| Control: C10-40 | 0.2222 | 22.2200 | 101773.7 | 100349.47 | 2.34 |
| Isoalkylamidopropyl- | | | 98940.7 | | |
| ethyldimonium | | | 100334 | | |
| Ethosulfate Rinsed | | | | | |
| Treatment Method I | 0.1688 | 16.8800 | 112436.53 | 110975 | 3.41 |
| | | | 109723.46 | | |
| | | | 110765 | | |
| Treatment Method II | 0.3102 | 31.0200 | 174707.6 | 179720.1 | 3.01 |
| | | | 190007.8 | | |
| | | | 174444.9 | | |

*Normalized to 1.00. All other results are relative to this control

Results as seen above clearly indicate that pre-treatment with a system containing Sodium Magnesium Silicate is effective in increasing the amount of C10-40 Isoalkylamidopropylethyldimonium Ethosulfate deposited on hair. These experiments indicate that this pre-treatment with a foundation containing sodium magnesium silicate can increase cationic deposition by 1.5 times versus a cationic treatment alone.

### Experiment 2:

In this second experiment, 50% gray hair tresses were washed with a 50% active solution of Sodium Laureth Sulfate (SLES) in water to remove any sub-stances from the hair surface. An untreated control was designated, dried and set aside. A second control tress was treated with 0.5 gram of a 1% solution of C10-40 Isoalkylamidopropylethyldimonium Ethosulfate, rinsed with water at 38° C. (+/−2.5° C.) at a flow rate of 60 ml/minute for fifteen seconds and dried.

The remaining tresses then underwent three types of treatment applications. In the first method of application, a hair tress was treated with 0.5 gram of the foundation composition of the present invention which was left on for three minutes. The tress was subsequently treated with 0.5 gram of a 1% solution of C10-40 Isoalkylamidopropylethyldimonium Ethosulfate, immediately rinsed with water as detailed above for fifteen seconds and dried.

In the second method of application, a hair tress was treated with 0.5 gram of the foundation composition of the present invention and immediately rinsed with water as detailed above for fifteen seconds. The tress was then treated with 0.5 gram of a 10% solution of C10-40 Isoalkylamidopropylethyldimonium Ethosulfate, immediately rinsed with water as detailed above for fifteen seconds and dried.

In the third method of application, a hair tress was treated with 0.5 gram of the foundation composition of the present invention and immediately treated with 0.5 gram of a 10% solution of C10-40 Isoalkylamidopropylethyldimonium Ethosulfate. The tress was then immediately rinsed with water as detailed above for fifteen seconds and dried.

All hair tresses were submitted to mass spectral quantification of C10-40 Isoalkylamidopropylethyldimonium Ethosulfate as detailed above. The results are in Table VI.

TABLE VI
(4 Injections of each extract)

| | | | | | % C10-40 |
|---|---|---|---|---|---|
| | | Injection | | | Isoalkyl- |
| | | Concentration, | | | amidopropyl- |
| | g of Hair | mg Hair | | Average | ethyldimonium |
| Description | Extracted | Extracted/ml | Peak Area | Peak Area | Deposited |
| Untreated Control | 0.2713 | 27.1300 | 50780.8000 | 46730.28 | *1.00 |
| | | | 46955.7000 | | |
| | | | 39752.4000 | | |
| | | | 49432.2000 | | |
| Control: C10-40 | 0.2537 | 25.3700 | 221864.0000 | 210938.50 | 4.83 |
| Isoalkylamidopropyl- | | | 182390.0000 | | |
| ethyldimonium | | | 218668.0000 | | |
| Ethosulfate Rinsed | | | 220832.0000 | | |
| Treatment Method 1 | 0.244 | 24.4000 | 367765.0000 | 387642.75 | 9.22 |
| | | | 336708.0000 | | |
| | | | 417961.0000 | | |
| | | | 428137.0000 | | |
| Treatment Method 2 | 0.3464 | 34.6400 | 411403.0000 | 410841.25 | 6.89 |
| | | | 314560.0000 | | |
| | | | 444653.0000 | | |
| | | | 472749.0000 | | |
| Treatment Method 3 | 0.3067 | 30.6700 | 427372.0000 | 442995.75 | 8.39 |
| | | | 400882.0000 | | |
| | | | 485721.0000 | | |
| | | | 458008.0000 | | |

*Normalized to 1.00. All other results are relative to this control

Results as seen above again indicate that pre-treatment with a system containing Sodium Magnesium Silicate increases the amount of C10-40 Isoalkylamidopropylethyldimonium Ethosulfate deposited on 50% gray hair, up to 1.9 times that of a system without a pretreatment.

### Experiment 3:

To evaluate the mode of action to deposit Sodium Magnesium Silicate, various types of systems were evaluated. Example systems are detailed in Table VII.

TABLE VII
Systems of Magnesium Silicate Deposition

| Spray System A | | | Spray System B | | | Shampoo C | | |
|---|---|---|---|---|---|---|---|---|
| Material | Function | % | Material | Function | % | Material | Function | % |
| Water | Solvent | 98.755 | Water | Solvent | 98.605 | Water | Solvent | 88.605 |
| Sodium | Binder, Film For- | | Sodium | Binder, Film For- | | Sodium | Binder, Film For- | |
| Magnesium | mer, Anionic Site | | Magnesium | mer, Anionic Site | | Magnesium | mer, Anionic Site | |
| Silicate | Enhancer | 1.000 | Silicate | Enhancer | 1.000 | Silicate | Enhancer | 1.000 |
| DMDM | Preservative | 0.238 | Calcium | Anionic Bridge | 0.150 | Calcium | Anionic Bridge | 0.150 |
| Hydantoin | | | Chloride | | | Chloride | | |
| Iodopropynl | Preservative | 0.007 | DMDM | Preservative | 0.238 | Sodium | Surfactant | 10.000 |
| Butycarbamate | | | Hydantoin | | | Laureth Sulfate | | |
| | | | Iodopropynl | Preservative | 0.007 | DMDM | Preservative | 0.238 |
| | | | Butylcarbamate | | | Hydantoin | | |
| | | | | | | Iodopropynl | Preservative | 0.007 |
| | | | | | | Butylcarbamate | | |

In this experiment, 50% gray hair tresses were washed with a 5% active solution of Sodium Laureth Sulfate (SLES) in water to remove any substances from the hair surface. An untreated control was designated, dried and set aside. A second control tress was treated with 0.5 gram of a 10% solution of C10-40 Isoalkylamidopropylethyldimonium Ethosulfate, rinsed with water at 38° C. (+/2.5° C.) at a flow rate of 60 ml/minute for fifteen seconds and dried.

The remaining tresses underwent treatment application with the above systems. With Spray System A, a hair tress was treated with 0.5 gram of the foundation composition of the present invention which was left on for three minutes. The tress was subsequently treated with 0.5 gram of a 1% solution of C10-40 Isoalkylamidopropylethyldimonium Ethosulfate, immediately rinsed with water as detailed above for fifteen seconds and dried. This test protocol was repeated using Spray System B.

Shampoo System C was applied to wet hair, worked into a lather for thirty seconds, rinsed for thirty seconds with water as detailed above. The tress was subsequently treated with 0.5 gram of a 1% solution of C10-40 Isoalkylamidopropylethyldimonium Ethosulfate, immediately rinsed with water as detailed above for fifteen seconds and dried.

All hair tresses were submitted to mass spectral quantification of C10-40 Isoalkylamidopropylethyldimonium Ethosulfate as detailed above. The results are in Table VIII. Please note that this test was conducted simultaneously with the second experiment and both control tresses are the same in the two experiments.

TABLE VIII
Injections of Each Extract

| | | | | | % C10-40 |
|---|---|---|---|---|---|
| | | Injection | | | Isoalkyl- |
| | | Concentration, | | | amidopropyl- |
| | g of Hair | mg Hair | | Average | ethyldimonium |
| Description | Extracted | Extracted/ml | Peak Area | Peak Area | Deposited |
| Untreated Control | 0.2713 | 27.1300 | 50780.800 | 46730.28 | *1.00 |
| | | | 46955.700 | | |
| | | | 39752.400 | | |
| | | | 49432.200 | | |
| Control: C10-40 | 0.2537 | 25.3700 | 221864.00 | 210938.50 | 4.83 |
| Isoalkylamidopropyl- | | | 182390.00 | | |
| ethyldimonium | | | 218668.00 | | |
| Ethosulfate, Rinsed | | | 220832.00 | | |
| Spray System A | 0.2883 | 28.8300 | 502145.00 | 557461.25 | 11.23 |
| | | | 631150.00 | | |
| | | | 594692.00 | | |
| | | | 501858.00 | | |
| Spray System B | 0.244 | 24.4000 | 367765.00 | 387642.75 | 9.22 |
| | | | 336708.00 | | |
| | | | 417961.00 | | |
| | | | 428137.00 | | |
| Shampoo System C | 0.3075 | 30.7500 | 279040.00 | 313269.75 | 5.91 |
| | | | 237690.00 | | |
| | | | 418624.00 | | |
| | | | 317725.00 | | |

*Normalized to 1.00. All other results are relative to this control

Results indicate additional benefits of using Spray System A which increases the deposition by 2.3 versus the C10-40 Isoalkylamidopropylethyldimonium Ethosulfate treatment alone.

### Experiment 4:

Further experiments were conducted to simulate stresses caused by everyday environmental exposure. This test also evaluated layering the active material, in this case C10-40 Isoalkylamidopropylethyldimonium Ethosulfate, either before or after application of the foundation composition. In this test, tresses underwent four categories of treatment, were exposed to UV irradiation using a Suntest XLS for three hours at 765 w/m2 irradiance and then quantified using a mass spectrometer. This experiment utilized 50% gray hair tresses pre-washed with a 5% active solution of Sodium Laureth Sulfate (SLES) in water to remove any substances from the hair surface.

The first category of treatment was designated as the untreated control. Tresses were washed with one milliliter of 5% active SLES solution for thirty seconds, rinsed with water at 38° C. (+/−2.5° C.) at a flow rate of 60 ml/minute for thirty seconds and dried. One milliliter of C10-40 Isoalkylamidopropylethyldimonium Ethosulfate solution was applied the tresses and then rinsed with water as detailed above for thirty seconds. The tresses were then exposed to UV irradiation as specified above, washed, also as specified above, with one milliliter of 5% active SLES solution and dried.

The second category of treatment was designed to test the spray foundation composition of the present invention. In this treatment, tresses were again washed with one milliliter of 50% active SLES solution for thirty seconds, rinsed with water as detailed above for thirty seconds and dried. One milliliter of C10-40 Isoalkylamidopropylethyldimonium Ethosulfate solution was applied the tresses and then rinsed with water as detailed above for thirty seconds. The tresses were then treated with one milliliter of the spray foundation composition, exposed to UV irradiation as specified above, washed, also as specified above, with one milliliter of 5% active SLES solution and dried.

The third category of treatment was designed to test the shampoo foundation composition. In this treatment, tresses were washed with one milliliter of Shampoo System C noted in the previous experiment for thirty seconds, rinsed with water as detailed above for thirty seconds and dried. One milliliter of C10-40 Isoalkylamidopropylethyldimonium Ethosulfate solution was applied the tresses and then rinsed with water as detailed above for thirty seconds. The tresses were exposed to UV irradiation as specified above, washed, also as specified above, with one milliliter of Shampoo System C and dried.

The fourth category of treatment was designed to test using both the shampoo foundation composition and the spray foundation composition. In this treatment, tresses were washed with one milliliter of Shampoo System C noted in the previous experiment for thirty seconds, rinsed with water as detailed above for thirty seconds and dried. One milliliter of C10-40 Isoalkylamidopropylethyldimonium Ethosulfate solution was applied the tresses and then rinsed with water as detailed above for thirty seconds. The tresses were then treated with one milliliter of the spray foundation composition, exposed to UV irradiation as specified above, washed, also as specified above, with one milliliter of 5% active SLES solution and dried.

All hair tresses were submitted to mass spectral quantification of C10-40 Isoalkylamidopropylethyldimonium Ethosulfate as detailed above. The results are in Table IX and Table X.

TABLE IX
Injection Series #1 (4 Injections of each extract) Day 1

| | | | | | % C10-40 |
|---|---|---|---|---|---|
| | | Injection | | | Isoalkyl- |
| | | Concentration, | | Average | amidopropyl- |
| | g of Hair | mg Hair | Response | Response | ethyldimonium |
| Description | Extracted | Extracted/ml | Factor | Factor | Deposited |
| Shampoo Foundation | 0.2065 | 20.6500 | 421444 | 388034.37 | 1.53 |
| Composition + Spray | | | 401099 | | |
| Foundation Composition | | | 392965 | | |
| | | | 386413 | | |
| | 0.1916 | 19.1600 | 382817 | | |
| | | | 377182 | | |
| | | | 373945 | | |
| | | | 368410 | | |
| Shampoo Foundation | 0.1863 | 18.6300 | 298616 | 285556.56 | 1.12 |
| Composition | | | 297521 | | |
| | | | 294576 | | |
| | | | 304930 | | |
| | 0.2131 | 21.3100 | 277042 | | |
| | | | 273247 | | |
| | | | 269506 | | |
| | | | 269015 | | |
| Spray Foundation | 0.2144 | 21.4400 | 324070 | 317357.47 | 1.25 |
| Composition | | | 321889 | | |
| | | | 320905 | | |
| | | | 318817 | | |
| | 0.2656 | 26.5600 | 318761 | | |
| | | | 313946 | | |
| | | | 310811 | | |
| | | | 309661 | | |
| Untreated Control | 0.224 | 22.4000 | 254785 | 254141.36 | 1.00 |
| | | | 253945 | | |
| | | | 252124 | | |
| | | | 250770 | | |
| | 0.2098 | 20.9800 | 252552 | | |
| | | | 250367 | | |
| | | | 248212 | | |
| | | | 270376 | | |

*Normalized to 1.00. All other results are relative to this control

TABLE X
Injection Series#2 (4 injections of each extract) Day 2

| | | | | | % C10-40 |
|---|---|---|---|---|---|
| | | Injection | | | Isoalkyl- |
| | | Concentration, | | Average | amidopropyl- |
| | g of Hair | mg Hair | Response | Response | ethyldimonium |
| Description | Extracted | Extracted/ml | Factor | Factor | Deposited |
| Shampoo Foundation | 0.2065 | 20.6500 | 319783 | 326309.84 | 1.60 |
| Composition + Spray | | | 322634 | | |
| Foundation Composition | | | 320376 | | |
| | | | 318642 | | |
| | 0.1916 | 19.1600 | 339849 | | |
| | | | 333223 | | |
| | | | 329807 | | |
| | | | 326165 | | |
| Shampoo Foundation | 0.1863 | 18.6300 | 281767 | 244964.52 | 1.20 |
| Composition | | | 278182 | | |
| | | | 268430 | | |
| | | | 265748 | | |
| | 0.2131 | 21.3100 | 220533 | | |
| | | | 217293 | | |
| | | | 214676 | | |
| | | | 213087 | | |
| Spray Foundation | 0.2144 | 21.4400 | 261727 | 260497.61 | 1.28 |
| Composition | | | 258673 | | |
| | | | 256542 | | |
| | | | 253997 | | |
| | 0.2656 | 26.5600 | 262590 | | |
| | | | 251113 | | |
| | | | 270262 | | |
| | | | 269078 | | |
| Untreated Control | 0.224 | 22.4000 | 209782 | 203514.10 | 1.00 |
| | | | 204350 | | |
| | | | 198897 | | |
| | | | 206460 | | |
| | 0.2098 | 20.9800 | 204154 | | |
| | | | 199742 | | |
| | | | 196600 | | |
| | | | 208128 | | |

*Normalized to 1.00. All other results are relative to this control

Results from this experiment indicate that C10-40 Isoalkylamidopropylethyldimonium Ethosulfate is substantive to hair when applied with either a spray foundation or a shampoo foundation or a combination of both. Even after UV irradiation and washing with a 5% active SLES solution, a significant amount of C10-40 Isoalkylamidopropylethyldimonium ethosulfate remains on hair. The combination of both a shampoo foundation and a spray foundation shows less loss of C10-40 Isoalkyamidopropylethyldimonium ethosulfate than either the spray foundation or shampoo foundation alone.

### Experiment 5:

It was hypothesized that the foundation composition alone protects the internal hair structure from degradation. To support this hypothesis, testing was conducted quantifying the amount of internal protein extracted from the hair.

In this test, medium brown hair tresses were pre-washed with a 5% active solution of Sodium Laureth Sulfate (SLES) in water to remove any substances from the hair surface. Half the tresses were set aside as untreated control tresses and the remaining half was treated with 0.5 grams of Spray Foundation Composition B per tress. All tresses were then dried.

The method used to determine the amount of protein extracted from hair is a modified Lowry assay using the following conditions. To analyze the protein content, 80 milligram samples were weighed and cut from each of the hair tresses and placed into individual flasks. To each flask, 10 milliliters of 0.04N Sodium Hydroxide solution was added and the hair samples were soaked for thirty minutes. One milliliter of Copper Carbonate solution (1 ml of 10% CuSO4+1 ml of 2% Potassium Tartrate+20 ml of 10% Na2CO3) was then added to each flask and soaked for fifteen minutes. Three milliliters of 0.18N Folin-Phenol solution (5 ml of 2N Folin-Phenol +50 ml of Water) was then added to each flask and soaked for forty additional minutes. Each resulting solution was filtered through a 0.45 μm nylon filter to remove any particles prior to analysis.

The UV-VIS Spectrophotometer was set up to measure wavelength 745 nm. The solution from each sample was measured to determine the absorption. Results are detailed in Table XI.

TABLE XI
Protein Analysis

| | Absorption | Standard | |
|---|---|---|---|
| | Results | Deviation | T test |
| Untreated Control | 0.1203 | 0.004 | 0.0380 |
| Tresses treated with Spray | 0.1130 | 0.001 | |
| Foundation Composition | | | |

As can been seen in Table XI, tresses treated with the foundation composition of the present invention had significantly less protein that was able to be extracted from the hair. These results support the hypothesis that the foundation composition is creating a protective layer on the surface of the hair which reduces degradation of the internal hair fiber by external forces.

### Experiment 6:

In order to evaluate the effect of layering material onto the surface of the hair and subsequently layering the foundation composition of the present invention, cationic deposition and removability was tested using Rubine Dye. The anionic Rubine dye (Direct Red 80) complexes with cationic materials deposited on the hair by the test product resulting in a pink color left on the hair.

In this test, piedmont hair tresses were washed with a 10% Sodium Lauryl Sulfate (SLS) solution to remove any initial substances from the hair. Three tresses were set aside for use as a control. The remaining tresses were each treated with one milliliter of a cationic conditioner formulated for normal hair which was left of the hair for one minute and then rinsed in water at 38° C. (+/−2.5° C.) with a flow rate of 60 ml/sec for one minute. Half of the tresses were then treated with the foundation composition of the present invention, 0.30 grams per hair tress and all tresses were subsequently dried.

To evaluate cationic removability, three tresses from each treatment group were set aside as unwashed and the remaining tresses from each treatment group were washed either 5, 10, 15 or 20 times. The wash procedure detailed that one milliliter of the 10% SLS solution was applied to each hair tress, agitated for one minute and rinsed for one minute in water as detailed above.

All hair tresses were then immersed in a 0.5% dye solution of Direct Red 80 per the formula detailed in Table XII for one minute per hair tress. Each tress was then rinsed in water at 25° C. for two minutes and thoroughly dried.

TABLE XII
Rubine Dye Solution

| Material | % |
|---|---|
| Deionized Water | 99.25 |
| Direct Red 80 | 0.50 |
| Glacial Acetic Acid | 0.25 |
| MEA | Adjust solution to pH 3.5 |

Each hair tresses was measured using a Minolta Spectrophotometer. The color measurement method of the Minolta Spectrophotometer mathematically computes and compares the color of the hair tresses. The method was developed by CIE (Commission Internationale de l'Eclairage) using L*, a* and b* values. Where, L* represents the difference between light (where L*=100) and dark (where L*=0). A* represents the difference between green (−a*) and red (+a*), and b* represents the difference between yellow (+b*) and blue (−b*). In this test, the a* values were evaluated to determine the degree of red deposition onto the hair fiber. Average results are detailed in Table XIII.

TABLE XIII
CATIONIC DEPOSITION AND REMOVABILITY RESULTS

| | Minolta a* values for tresses | | | | |
|---|---|---|---|---|---|
| | at each number of washes | | | | |
| | 0 | 5 | 10 | 15 | 20 |
| Untreated Control | 9.617 | n/a | n/a | n/a | n/a |
| Treated with conditioner | 17.270 | 11.317 | 10.760 | 10.433 | 9.683 |
| only | | | | | |
| Treated with conditioner | 19.837 | 13.123 | 11.907 | 11.783 | 9.903 |
| and foundation composition | | | | | |
| treatment | | | | | |
| T test comparing hair | 0.006 | 0.044 | 0.060 | 0.020 | 0.793 |
| treatments | | | | | |

As is evident in the results detailed in Table XIII, all hair tresses treated with cationic conditioner had significantly more red deposition then the untreated tresses. As shown in the data provided, hair tresses that were treated with the foundation composition treatment on top of the cationic conditioner had significantly more red deposition than those treated only with conditioner. Furthermore, the cationic deposition remains more intense, as is apparent in the higher a* results, through 15 washings when the foundation composition of the present invention has been applied on top of the cationic conditioner.

### Experiment 7:

To test simultaneous action whereby an active material is deposited in the same step as the foundation composition, UV absorbers were added to the foundation composition, exposed to UV light and analyzed for hair damage using infrared spectroscopy.

In this experiment, 100% gray hair tresses were washed with a 10% Sodium Laureth Sulfate (SLES) solution to remove any initial substances from the hair. Two control tresses were set aside, one to be the undamaged control and the other to be exposed to UV. The test tress was conditioned and shampooed using market products containing no UV absorbers. The tress was then treated with three grams of the foundation composition containing 0.1% of a known UV absorbing silicone and dried.

The test tress and the UV exposed control tress were exposed to UV using a Suntest XLS set to 765 w/m2 irradiance for 98 hours. During this exposure time, hair tresses were flipped over every seven hours to ensure even distribution of the irradiation. Every fourteen hours, tresses were re-washed: the UV exposed control tress was washed with SLES and the test tress was washed with market products followed by three grams of the foundation composition.

Following UV exposure, the hair tresses were washed with 10% Sodium Lauryl Sulfate solution and Isopropyl Alcohol until any residual product was removed from the surface of the hair. The tresses were then thoroughly dried. The IR spectra was recorded using a Nicolet 380 FTIR with ATR accessory at resolution of 4 ^{cm-1} and 32 scans. This data was analyzed to determine the ratio of the reflectance values between Cysteic Acid and Cystine S-monoxide. The size of the SO₃H band near 1040 cm⁻¹ increases when the hair is damaged. The band near 1075 cm⁻¹ represents Cystine S-monoxide, which is an intermediate oxidation product before Cystine is further oxidized to Cysteic Acid. The ratio between these two spectra is an indicator for hair damage.

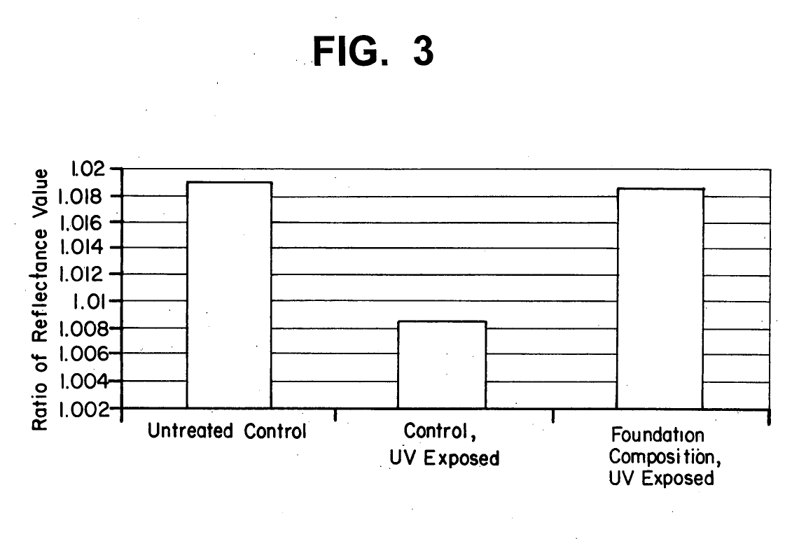

Each hair sample was recorded three times at top, middle and end for a total of nine points per sample. The results are shown in FIG. 3.

As can be seen in reflectance ratios, the hair treated with the foundation composition does not show significant signs of damage after 98 hours of UV exposure compared to the untreated control and the UV exposed control. This indicates that the foundation composition is simultaneously depositing the sunscreen onto the hair, and working to prevent damage to the hair fiber.

### Experiment 8:

To further test the applicability of this invention, various styling polymers were evaluated to determine if the addition of the foundation composition impacted their style retention over time. In this experiment, medium blonde tresses 11 centimeters in length were pre-washed with a 5% active solution of Sodium Laureth Sulfate (SLES) in water to remove any substances from the hair surface.

Tresses that were undergoing the experimental treatment were treated with 0.5 grams of the foundation composition. All tresses were then treated with one of three styling polymers: VP/DMAPA Acrylates Copolymer, Polyquaternium-11 or Polyquaternium-46. To further evaluate the tresses, half of the control and treated tresses were then rinsed for fifteen seconds in water at 38° C. (+/−2.5° C.) at a flow rate of 60 ml/minute.

All tresses were then rolled into curlers and allowed to fully dry. The rollers were removed and tresses were clipped onto measuring boards. Tresses were evaluated initially, hung for five hours at 23° C. and 21% relative humidity, then combed ten times per tress with fine tooth combs and re-evaluated.

The percent curl retention values were calculated using the formula: ((L−Lt)/(L−Lo))×100 where L Length of the hair fully extended (11 cm), Lo=Length of the treated hair curl and Lt=Length of the hair curl after combing “t” times, where “t” equals 10. The results are shown in Table XV.

The percent difference in curl retention was calculated using the formula: ((|x1−x2|)/((x1+x2)/2))×100 where x1=Control % curl retention and x2=Treated % Curl Retention. The results are shown in Table XVI.

TABLE XV
Percent Curl Retention

| | | | | Treated with |
|---|---|---|---|---|
| | | Treated with | | Foundation |
| | | Foundation | Control, | Composition, |
| | Control | Composition | Rinsed | Rinsed |
| VP/DMAPA Acrylates | 89.29 | 89.47 | 67.14 | 84.51 |
| Copolymer | | | | |
| Polyquaternium-11 | 80.52 | 88.31 | 59.72 | 64.38 |
| Polyquaternium-46 | 81.25 | 84.62 | 76.06 | 84.29 |

TABLE XVI
Percent Difference in Curl Retention

| | Unrinsed Tresses | Rinsed Tresses |
|---|---|---|
| VP/DMAPA Acrylates | 0.21 | 22.90 |
| Copolymer | | |
| Polyquaternium-11 | 9.23 | 7.51 |
| Polyquaternium-46 | 4.06 | 10.26 |

As can be seen in the data, the results indicate applying the foundation composition increases the style retention of various polymers. While the results vary, especially in the un-rinsed application, this data shows a trend of improved performance by the styling polymers. This data is especially noted in the tresses that have been rinsed, where the polymer function is significantly greater in two of the three polymers tested. This indicates that the foundation composition binds the styling polymers to the hair and resists removal with water.

It will thus be seen that the objects set forth above, among those made apparent from the preceding description, are efficiently entertained and, since certain changes may be made in carrying out the above process, and in the compositions set forth without departing from the scope of the invention, it is intended that all matter contained in the above description shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

Particularly, it is to be understood that in said claims, ingredients or compounds recited in the singular are intended to include compatible mixtures of such ingredients wherever the sense permits.

Having described our invention, what we claim as new and desire to secure up by Letters Patent is:

1. A hair styling foundation composition formulated to enhance performance of additional compositions and provide a protective film to hair fibers, said composition comprising between about 0.05% and 5% by weight based upon the weight of the entire composition of sodium magnesium silicate and water forming the balance.

2. The hair styling foundation composition defined in claim 1, wherein said composition further comprises between about 0.05% and 1.5% by weight based upon the weight of the entire composition of a preservative.

3. The hair styling foundation composition defined in claim 2, wherein said composition further comprises one or more additives selected from the group consisting of thickeners, suspension aids, silicones, humectants, emulsifiers, calcium ion sources, solubilizers, fragrances, and pH adjusters.

4. The hair styling foundation composition defined in claim 3, wherein said composition is further defined as comprising up to 5% by weight based on the weight of the entire composition of a thickener, up to 5% by weight based on the weight of the entire composition of a suspension aid, up to 10% by weight based upon the weight of the entire composition of a silicone, up to 15% by weight based upon the weight of the entire composition of a humectant, up to 2.5% by weight based upon the weight of the entire composition of solubilizer, up to 1% by weight based upon the weight of the entire composition of a fragrance, and up to 3% by weight based on the weight of the entire composition of a pH adjuster.

5. The hair styling foundation composition defined in claim 4, wherein the preservative comprises one or more selected from the group consisting of DMDM Hydantoin, Iodopropynyl Butylcarbamate, Methylparaben, Propylparaben, Methylchloroisothiazolinone, Methylchloroisothiazolinone, Diazolidinyl Urea, Imidazolidinyl Urea.

6. The hair styling foundation composition defined in claim 4, wherein the thickeners/suspension aids comprise one or more selected from the group consisting of Carbomer, Acrylamide/Sodium Acryloyldimethyltaurate/Acrylic Acid Copolymer, Acrylates/Ceteth-20 Itaconate Copolymer, Sodium Polyacrylate, Polyacrylamide, Hydrogenated Polydecene, Hydroxyethyl-cellulose, Xanthan Gum, Carrageenan, and Guar.

7. The hair styling foundation composition defined in claim 4, wherein the silicones comprise one or more selected from the group consisting of PEG/PPG-15/15 Dimethicone, PEG/PPG-18/18 Dimethicone, PEG-12 Dimethicone, PEG/PPG-17/18 Dimethicone, Lauryl PEG/PPG-18/18 Methicone, PEG/PPG-20/6 Dimethicone, PEG/PPG-4/12 Dimethicone, PEG/PPG-20/20 Dimethicone, Dimethicone, Amodimethicone, and Polysilicone-15.

8. The hair styling foundation composition defined in claim 4, wherein the humectants comprise one or more selected from the group consisting of Glycerin, Sorbitol, Propylene Glycol, Butylene Glycol, Rice Oil Glycereth-8 Esters, Acetamide MEA, Hydroxyethyl Urea, and Methyl Gluceth-20.

9. The hair styling foundation composition defined in claim 4, wherein the solubilizers comprise one or more selected from the group consisting of PPG-5-Ceteth-20, Trideceth-6, Laureth-7, Polysorbate 20, Polysorbate 40, Polysorbate 80, PEG-40 Hydrogenated Castor Oil, and PEG-60 Hydrogenated Castor Oil.

10. The hair styling foundation composition defined in claim 4, wherein the emulsifiers comprise one or more selected from the group consisting of Cetyl Alcohol, Stearic Acid, Glyceryl Stearate, PEG-100 Stearate, Stearyl Alcohol, Cetearyl Alcohol, Cetyl Esters, Palmitic Acid and Lecithin.

11. The hair styling foundation composition defined in claim 4, wherein the pH adjustors comprise one or more selected from the group consisting of Aminomethyl Propanol, Triethanolamine, Citric Acid, EDTA, Disodium EDTA, Tetrasodium EDTA, Trisodium EDTA, Sodium Hydroxide, and Phosphoric Acid.

12. The hair styling foundation composition defined in claim 3, wherein said composition is formulated as a spray composition.

13. The hair styling foundation composition defined in claim 12, wherein said spray composition further comprises up to 5% by weight based upon the weight of the entire composition of one or more selected from the group consisting of protein, vitamins, herbals, and UV absorbers.

14. The hair styling foundation composition defined in claim 13, wherein said spray composition further comprises up to 10% by weight based upon the weight of the entire composition of one or more polymers, and up to 2% by weight based upon the weight of the entire composition of a calcium ion source.

15. The hair styling foundation composition defined in claim 3, wherein said composition is formulated as a shampoo.

16. The hair styling foundation composition defined in claim 15, wherein said composition further comprises between about 5% and 20% by weight based upon the entire weight of the composition of a primary surfactant, between about 0.2% and 20% by weight based upon the weight of the entire composition of a secondary tertiary and additional surfactant, up to 5% by weight based upon the weight of the entire composition of one or more selected from the group consisting of proteins, vitamins, herbals and UV absorbers, up to 10% by weight based upon the weight of the entire composition of one or more polymers and up to 2% by weight based upon the weight of the entire composition of a calcium ion source.

17. A process for providing a film to hair fibers which enhances the performance of additional compositions and provides a protective film to the hair fibers, said process comprising the steps of:
A. forming a hair styling foundation composition comprising between about 0.05% and 5% by weight based upon the entire weight of the composition of sodium magnesium silicate and water forming the balance; and
B. applying the hair styling foundation composition to the hair fibers by layering the composition on the hair fibers.

18. The process defined in claim 17 comprising the additional steps of:
C. applying one or more supplemental products selected from the group consisting of styling compositions and conditioning agents to the hair by layering the styling compositions and agents on the hair fibers.

19. The process defined in claim 18, wherein the supplemental products are further defined as comprising cationic compositions which interact with the anionic sites created by the foundation composition.

20. The process defined in claim 18, wherein the supplemental cationic compositions are applied to the hair prior to the application of the hair styling composition.

21. The process defined in claim 19, wherein the supplemental cationic compositions and the hair styling foundation are applied simultaneously to the hair fibers.

22. The process defined in claim 17, wherein said hair styling foundation composition is formulated as a spray composition by incorporating up to 5% by weight based upon the weight of the entire composition of one or more selected from the group consisting of protein, vitamins, herbals, and UV absorbers, and up to 10% by weight based upon the weight of the entire composition of one or more polymers, up to 2% by weight based upon the weight of the entire composition of a calcium ion source, and up to 5% by weight based on the weight of the entire composition of a thickener, up to 5% by weight based on the weight of the entire composition of a suspension aid, up to 10% by weight based upon the weight of the entire composition of a silicone, up to 15% by weight based upon the weight of the entire composition of a humectant, up to 2.5% by weight based upon the weight of the entire composition of solubilizer, up to 10% by weight based upon the weight of the entire composition of a fragrance, and up to 3% by weight based on the weight of the entire composition of a pH adjuster.

23. The process defined in claim 17, wherein said hair styling foundation composition is formulated as a shampoo by incorporating between about 5% and 20% by weight based upon the entire weight of the composition of a primary surfactant, between about 0.2% and 20% by weight based upon the weight of the entire composition of a secondary tertiary and additional surfactant, up to 5% by weight based upon the weight of the entire composition of one or more selected from the group consisting of proteins, vitamins, herbals and UV absorbers, up to 10% by weight based upon the weight of the entire composition of one or more polymers, up to 2% by weight based upon the weight of the entire composition of a calcium ion source, up to 5% by weight based on the weight of the entire composition of a thickener, up to 5% by weight based on the weight of the entire composition of a suspension aid, up to 10% by weight based upon the weight of the entire composition of a silicone, up to 15% by weight based upon the weight of the entire composition of a humectant, up to 2.5% by weight based upon the weight of the entire composition of solubilizer, up to 1% by weight based upon the weight of the entire composition of a fragrance, and up to 3% by weight based on the weight of the entire composition of a pH adjuster.
